(19) United States
(12) Patent Application Publication
(10) Pub. No.: US 2003/0118671 A1
(43) Pub. Date: Jun. 26, 2003

(54) Formulation containing peanut leaf extract and its preparation

(76) Inventors: Qiaochu Wang, Shanhai (CN); Chuanyu Pang, Shanghai (CN); Shangiian Du, Shanghai (CN); Ming Shi, Shanghai (CN); Jian Xu, Shanghai (CN); Xiaofeng Zhang, Shanghai (CN)

Correspondence Address: CHRISTIE, PARKER & HALE, LLP, 350 WEST COLORADO BOULEVARD, SUITE 500, PASADENA, CA, 91105, US

(21) Appl. No.: 10/148,732

(22) PCT Filed: Dec. 4, 2000
(86) PCT No.: PCT/CN00/00530

(30) Foreign Application Priority Data
Dec. 2, 1999 (CN) 99124202.5

(51) Int. Cl. A61K 35/78

(52) U.S. Cl. 424/725; 424/774; 424/779

(57) ABSTRACT

The present invention relates to a pharmaceutical preparation of natural plants. More particularly, the present invention relates to a preparation containing leaf extract of Arachis hypogaea and process for making the same.

### FIELD OF THE INVENTION

The present invention relates to a pharmaceutical preparation of natural plants. More particularly, the present invention relates to a preparation containing leaf extract of Arachis hypogaea and process for making the same.

### BACKGROUND OF THE INVENTION

Leaves of A. hypogaes, the part of Arachis hypogaea which growing on the ground, contain many kinds of volatile components. Since 1970s, studies on leaves of A. hypogaea have been reported. For example, the chemical structures of 8 compounds, most of which are terpene alcohols, from leaf extract of A. hypogaea were identified by S. E. Young et al (DELAHOMA university, US) in 1973 by means of steam distillation, ether extraction and gas chromatograph analysis (Phytochemistry 12;950, 1973); the effects of leaf extract of A. hypogaea in ethanol, petroleum ether and water on sedative-hypnotic action were studied by L. I. Guofeng et al (Chinese GuangXi Youjiang Nationalities Medical College) in 1987. The results of these studies show that leaf extract of A. hypogaea has sedative-hypnotic effect on mice, with less toxicity broader safe limits, and convenient administration (Chinese Traditional and herbal drugs 18(2): 70, 1987). However, no report hereinbefore related to the separation of effective components and the fractions effective for the treatment of insomnia has been disclosed.

### SUMMARY OF THE INVENTION

One object of the present invention is to separate the effective fractions for the treatment of insomnia from leaf extract of A. hypogaea and further, to separate and identify the effective components therefrom.

Thus, the present invention provide a process for extracting and separating leaf extract of A. hypogaea, and a process for producing the preparation containing leaf extract of A. hypogaea.

Another object of the present invention is to provide the preparation containing leaf extract of A. hypogaea, which is composed of the effective components of leaf extract of A. hypogaea and pharmaceutical carrier or excipient, and wherein the effective components of leaf extract of A. hypogaea and the pharmaceutical carrier or excipient are present in 100% said preparation at any ratio of 1˜85% effective leaf extract of A. hypogaea to 99˜15% pharmaceutical carrier or excipient.

The resource of stem and leaves of A. hypogaea is very abundant in China. The optimal time for its harvest is from August to October. The pharmaceutical application of leaves of A. hypogaea was seldom reported in ancient literatures. Although a few records related to this have been described in some modern folk simple prescriptions, most of them are not in detail. Further, neither the property nor the function of leaves of A. hypogaea has been determined. During collecting and tidying folk simple prescription herbals, remembering what had been observed in childhood rural life, the present inventor found that there may be some relationship between the human sleeping function and the biologic characters of leaves of A. hypogaea, which are consistent with the nature Yin-Yang decreasing and increasing rule of ‘opening in the day and closing in the night’. Therefore, since 1988, stem and leaves of A. hypogaea have been adopted to prepare ‘Luo Hua An Shen mixture’, and the effect thereof has been studied in experiment. As the result, stem and leaves of A. hypogaea are found effective to treat insomnia; additionally, stem and leaves of A. hypogaea are useful in reducing blood pressure, and thus could treat hypertension.

The present leaf extract of A. hypogaea (Luo Hua An Shen mixture) exhibited remarkable therapeutic effects on insomnia, and also has enhanced immunological function, improved cerebral blood vessel function and good clinical efficacy. The present process is simple, and is fit for extensive manufacture. The present preparation containing leaf extract of A. hypogaes can be used in the form of oral liquid, capsule, tablet, granule, diluent or powder.

### BRIEF DESCRIPTION OF THE DRAWINGS

FIG. 1 shows a schematic view for explaining the comparative effects on correct response of experimental animals between control group and administration group after 7 days of formal training and learning.

### DETAILED DESCRIPTION OF THE INVENTION

The present invention provided a process for extracting and separating leaf extract of A hypogaea, comprising pulverizing leaves of A. hypogaea, extracting twice with adequate boiling water, filtering, combining the extract and then concentrating to form the extractum, with a yield of about 10% (approximately 50% water contained in the extractum), mixing the resulting extractum with inert material uniformly, and then triturating after dried in 70° C., charging the resultant into Soxhlet's extractor and extracting with petroleum ether, ethyl acetate, acetone and anhydrous ethanol successively, or extracting and separating the resultant with varied specific solvent depending on the compound contained in the leaf of A. hypogaea, to collect the fractions containing different kinds of compounds. A: total extract; B: protein, C. polysaccharide, D: flavone, tannin, E: lipoclastic, volatile oil.

Pharmacological activities of these components were studied The results are described below.

Spontaneous behavior of the mouse was determined by photoelectric test. The result was shown in Table 1.

Dose of administration was corresponded to 1.2 g crude drugs per 10 g body weight (if not indicated otherwise, the same meaning hereinafter), administered orally

TABLE 1
Influence of 5 kinds of extracts on spontaneous
movement of mice (t value)

| | Type of | | | | | |
|---|---|---|---|---|---|---|
| | compound | | | | | |
| | contained | | t value after administration | | | |
| Group | in extract | n | 0-15′ | 16-30′ | 31-45′ | 46-60′ |
| A | Total extract | 4 | 0.49 | 0.15 | 0.55 | 0.62 |
| B | protein | 6 | 0.40 | 0.81 | 1.45 | 1.71 |
| C | polysaccharide | 6 | 0.99 | 0.90 | 1.08 | 1.18 |
| D | flavone, | 6 | 0.79 | 1.01 | 1 65 | 1.58 |
| | tannin | | | | | |
| E | liposoluble, | 6 | 0.83 | 1.80 | 2.22 | 2.12 |
| | volatile oil | | | | | |
| normal | | 6 | | | | |
| saline(s.c) | | | | | | |

As shown in Table 1, fraction E decreases the spontaneous movement of mice significantly, and is more effective than other fractions. Accordingly, extensive studies were conducted on this fraction. The result was shown in Table 2.

TABLE 2
Influence of fraction E on spontaneous
movement of mice (t value)

t value after administration

| Group | n | 0-15′ | 16-30′ | 31-45′ | 46-60′ |
|---|---|---|---|---|---|
| E | 10 | 2.25 | 3.15** | 1.21 | 0.196 |
| (s.c) | 10 | | | | |

As shown in Table 2, the spontaneous movement of mice is significantly or very significantly decreased within 0 to 30 minutes after administration.

Fraction E was further analyzed by gas-infrared analysis. Table 3 reports the test results.

TABLE 3
Gas-Infrared analysis

| | Retain | | Name of compound | | | | | |
|---|---|---|---|---|---|---|---|---|
| Serial | Value | amount % | VB Value* | compound A | VB Value | Compound B | VB Value | Compound C |
| 1 | 5.24 | 0.446 | 0.24 | ethyl acetate | 0.33 | methyl acetate | 0.34 | propyl acetate |
| 2 | 7.37 | 1.310 | 0.17 | dlbutyl ether | 0.22 | dibutyl ether | 0.23 | tri-dimethanol |
| 3 | 12.08 | 1.356 | 0.42 | dlethylamino | 0.44 | 2-butoxy-ethyl | 0.45 | 4,4′-dimethanol- |
| | | | | accetic acid ethyl | | hexadiacid | | butyric acid |
| | | | | ester | | | | |
| 4 | 22.40 | 2.976 | 0.13 | 5-heptylene-8 | 0.30 | pentane-2-one | 0.30 | hexone-ethyl |
| | | | | | | | | methyl-2-one |
| 5 | 41.38 | 4.698 | 0.15 | citral | 0.39 | D-carvone | 0.40 | L-carvone |
| 6 | 43.73 | 7.149 | 0.10 | citral** | 0.40 | D-carvone | 0.41 | L-carvone |

Note: * VB value is the matching coefficient which indicates the similarity degree between the resultant spectrum of the detected substance and the standard spectrum stored in computer. The smaller of the VB value, the higher of the reliability

** Peaks 5 and 6 indicate the existence of citral, which may be present in form of isomers.

Conclusion:

(1) Evaluated on the pharmacological activity, fraction E was confirmed having the positive effects on the sedative-hypnotic.

(2) Linalool, as a known chemical component having the positive effects on the sedative-hypnotic, was proved to be contained in the fraction E by gas-infrared analysis as shown in peak 8.

The present invention also provided a process for making said preparation containing leaf extract of A. hypogaea. Leaves of A. hypogaea was dried, pulverized and passed through sieve of 10-40 meshes prior to being charged into a multifunctional reaction vessel equipped with a heating jacket and reflux device. 144 parts leaves were heated under reflux with 200-1000 parts non-polar solvent (ether, petroleum ether) for 1˜3 times, 2˜4 hours for each time. Additionally, steam distillation can be utilized for extracting liposoluble fraction A. The residual leaves were extracted again with 200-1200 parts polar solvent (ethanol, water) for 1˜3 times, 1˜4 hours for each time. After removing the solvents, the two extracted solutions were combined with each other The resultant was made into oral liquid, capsule, tablet, granule, diluent or powder after adding. 0˜2% flavoring agent, 0˜1% antiseptic and 0˜10% other pharmaceutical carrier or excipient according to prescription.

The Method for Quality Control of the Present Preparations:

1. Sample Liquid of the Present Preparation

[Qualitative Identification]

2 g preparation containing leaves of A. hypogaea was dissolved into 10 ml water, and then extracted twice with 5 ml ether using separatory funnel combined the ether solutions, and concentrated to 2 ml, the resultant was used as the test sample for examination and thin-layer chromatography analysis.

1 ml bromine water was added into said ether extracted solution and shaken. The color of bromine water turned to gray from red. A little flocculent solid would emerge along with the volatilization of ether.

2. Preparation of Control Medicinal Herb and Compound Sample

2 g control leaves of A. hypogaea were immerged into 20 ml ether for 1 hour and filtered. The filtered solution was evaporated to dryness. Then the residue was extracted twice with 10 ml ether. Combined the ether solution, and then concentrated to 2 ml, which was used as control medicinal herb solution.

1 mg standard compounds of Linalool and citral were dissolved into 2 ml ether, which was used as control solution.

3. Thin-layer Chromatography Examination

Silica gel thin-layer was prepared according to Pharmacopoeia of the People's Republic of China, Part 1, appendix VIB, 1995. 2 μl test sample, medicinal herb solution and control solution were applied to the initial line of the same thin-layer plate, respectively. The thin-layer plate was developed with petroleum ether/ethyl acetate (10:1) mixture (upper development) and the color was displayed by iodine or FeCl₃-K₄Fe(CN)₆ The spots with the same R_{f} value as that of standard control sample should been displayed both on the test sample and the medicinal herb sample.

[Examination]

The method utilized herein is conformity with the regulations set forth in the appendix of pharmacopoeia (see Pharmacopoeia of the People's Republic of China, Part 1, Appendix I_{B}, I_{C}, I_{D}, I_{J}, I_{L,} 1995).

[Quantitative Determination]

1. Volatile Oil

Volatile oil should be present in an amount of 0.01˜0.1%, determined by the method described in Pharmacopoeia of the People's Republic of China, Part 1 Appendix XD, 1995.

The component of volatile oil was analyzed using thin-layer chromatography (see Pharmacopoeia of the People's Republic of China, Part 1, Appendix VIB, 1995) or gas chromatograph (see Pharmacopoeia of the People's Republic of China, Part 1, Appendix VIE, 1995). Linalool and citral should be detected.

Citral content>0.01 mg/g crude drugs, Linalool content>0.001 mg/g crude drugs.

2. Choline

Choline content was determined using high performance liquid chromatography according to Pharmacopoeia of the People's Republic of China, Part 1, Appendix VID, 1995.

Choline content>0.02 mg/g crude drugs.

The present invention is illustrated with reference to the following examples related to making the preparation containing leaf extract of A. hypogaea and to the experiments thereof.

### EXAMPLES

### Example 1

Oral Liquid

100 g dried leaves of A. hypogaea were pulverized to coarse powders (10 meshes), The coarse powders were immersed into 5 times of water for 24 hours and extracted by steam distillation until the distillate became transparent liquid. The distillate was cooled, and then stored hermetically. The residual leaves were boiling for 2 hours with 2 times water and filtered. The filter residue was subjected to extraction as described above. Combined the filtrates and vacuum concentrated to a specific density of 1.2. The concentrated solution was mixed with 3 times of 95% by volume ethanol and vacuum filtered after standing for 12 hours. Ethanol was recycled after removing the filter residue. The concentrated liquid was added 5% sucrose, 1% sodium cyclamate, and then mixed with the volatile oil. The volume was adjusted to 3 g crude drug/ml. The resulting liquid was bottled (10 mL/bottle) and sterilized by boiling, thereby obtained the oral liquid of leaves of A. hypogaea.

### Example 2

Granule

100 g dried leaves of A. hypogaea were pulverized to coarse powders (10 meshes). The concentrated liquid with a density of 1.1 was prepared according to Example 1 and was dried by spray drying, added 5% dextrin thereto and dry granulated. Then the resultant was mixed with the volatile oil and packed hermetically, thereby obtained the granule of leaves of A. hypogaea.

### Example 3

Tablet

100 g dried leaves of A. hypogaea were pulverized to coarse powders (10 meshes). The coarse powders were immersed into 5 times of petroleum ether for 24 hours under 60-90° C., and then filtered. After removing solvent from the filtrate, the liposoluble fraction was obtained and stored. The residual leaves were extracted under reflux twice (2.5 hours for each time) with 5 times of 70% ethanol after removing petroleum ether by evaporation, and then filtered. Combined the filtrates. Ethanol was recovered by vacuum concentration till density of the concentrated filtrate reached 1.4. The concentrated filtrate was dried by vacuum, pulverized and passed through sieve of 60 meshes, mixed with 5% starch, 5% microcrystalline cellulose, and 1% magnesium stearte, and then granulated. Thus obtained granules were mixed with the liposoluble fractions and tabletted, encapsulated with plastic, and packed.

### Example 4

Capsule

The granules obtained from Example 3 were encapsulated in capsules, encapsulated with plastic and packed, thus obtained the capsule of leaves of A. hypogaea.

### Example 5

Powder

The product obtained from Example 2 was granulated, pulverized and passed through sieve no.6, encapsulated and packed, thus obtained the powder of leaves of A. hypogaea.

### Experiment Example 1

Effect of the Present Preparation Containing Leaf Extract of A. hypogaea (Luo Hua An Shen Mixture II) on Electrical Activity of Cerebral Cortex in Rabbit

Materials and Methods:

Animals: male or female adult New Zealand rabbits.

Drugs: Luo Hua An Shen mixture II, provided by Huangshan Pharmaceutical Factory; Crystal N provided by Department of Chemistry of Communication University; 4 mL. Luo Hua An Shen mixture (equivalent to 12 g crude drug) was administrated orally for each rabbit.

Methods: Stainless steel electrodes were placed in the rabbit head with surgery on several days before experiment. EEG and respiration were recorded with Nihon Kohden 8 channels polygraph system. Hypnosis action was evaluated by Sigma and Delta indexes that are recognized in the world.

Results: Comparing with saline control group, the Sigma indexes of the group which 4 mL Luo Hua An Shen mixture was administrated orally were increased significantly, the Delta indexes were increased at the same time (Table 4). Comparing with pre-administration, both Sigma and Delta indexes were increased after administration. The actions were appeared in about 45 minutes and lasted for 2˜3 hours (see Table 5).

Respiration became slow and smooth at the same time when hypnosis appeared. Respiratory rhythm decreased from 128.6±15.3 times/min (N=7) in pre-administration to 116.4±12.8 times/min after 1 hour and to 103.3±13.8 times/min after 2 hour.

No abnormal brain wave was recorded from cerebral cortex of treated adult rabbits

TABLE 4
Effect of Luo Hua An Shen mixture II on Sigma
and Delta indexes of cerebral cortex in rabbit

| Group | Sigma Index | Delta Index |
|---|---|---|
| S.C. | 6.2 ± 0.9 | 2.4 ± 1.1 |
| | (N = 6) | (N = 6) |
| LuoHua An Shen mixture II | 8.9 ± 1.2* | 2.7 ± 0.5 |

*P < 0.05

TABLE 5
Effect of Luo Hua An Shen mixture II on Sigma
and Delta Index at pre-and post-administration

| | pre- | post-administration | | |
|---|---|---|---|---|
| Index | administration | 1 hr | 2 hr | 3 hr |
| Sigma index | 4.8 ± 1.4 | 9.9 ± 1.3** | 9.5 ± 0.8** | 8.4 ± 1.9** |
| (N = 8) | | | | |
| delta index | 1.7 ± 0.5 | 2.6 ± 0.4** | 2.4 ± 0.4** | 1.9 ± 1.0 |
| (N = 8) | | | | |

*P < 0.05
**P < 0.01

### Experiment Example 2

Effect of Luo Hua An Shen Mixture II on Arousal Level in Rabbit

Materials and methods: the same animals and drugs were used in this example, drugs at dose of 4 ml were delivered by single dosage orally.

Methods: One extremitie of rabbit was bound with a fixed sensor(wrist), which was connected to the newest special microcomputer mini-logger 2000 (US). Activities which indicating the arousal level of rabbits in 24 hours were recorded by the microcomputer. The results prior to and posterior to administration were compared. See Table 6.

TABLE 6
Effect of Luo Hua An Shen mixture II on arousal level in rabbit

| | pre-administration | post-administration (1 hr) |
|---|---|---|
| arousal level (index) | 20.1 ± 7.6 | 13.4 ± 5.4* |
| (N = 8) | | |

*P < 0.05

The results show that the indexes decreased significantly within 2 to 3 hours after administration. Therefore, there was a remarkable different of indexes between prior to and posterior to administration.

### Experiment Example 3

Effect of the Present Preparation Containing Leaf Extract of A. hypogaea (Luo Hua An Shen Mixture I) on Isolated Basal Artery of Pigs

Materials and Methods:

Drugs: Luo Hua An Shen mixture I (non-volatile component) was provided by Shanghai Huangshan Pharmaceutical Factory, lot number 970129. Volatile component was provided by Shanghai Huangshan Pharmaceutical Factory. Danshen Injection (S. Miltiorrhiza) provided by Shanghai Ninth Pharmaceutical Factory, lot number 970301. Injection of phenylephrine was provided by Shanghai 11^{th} Pharmaceutical Factory, lot number 811223.

Instruments: Mono-pen recorder (manufactured by Shanghai Automatization and Instrument Factory) Tonotransducer (provided by the pharmacological teaching and research group of Shanghai Employee Medical College)

Methods: Isolated pig basal arteries were obtained from Shanghai Longhua Meat processing Plant. Basal artery was removed from occipital magnum foramen with forceps immediately after cutting off the head of pigs. Blood was washed out with Krebs-Ringer solution quickly. Then basal arteries were put into a thermos bottle bubbled with oxygen as soon as possibly Basal arteries were placed in a double layers tissue bath filled with 37±0.5° C. Krebs-Ringer solution under continuous bubbling of oxygen. 1 g substances were loaded on the isolated tissues that was connected to the tonotransducer, and then recorded by mono-pen recorder. The solution was changed every 30 minutes. Experiments were performed after the basal arteries were equilibrated for 1.5 hours. The tissues were washed three times after administration of drug each times. Other drugs were delivered after equilibration. The results were shown in Table 7.

TABLE 7
Effect of drugs on isolated basal artery of pigs

| 10⁻³ M | | | | |
|---|---|---|---|---|
| phenylephrine | n | Test sample | Drug | 10⁻³ M phenylephrine |
| 5.88 ± 2.22 | 8 | Luo Hua An Shen mixture I 3% | −4.4 ± 2.85 | 1.44 ± 1.29 |
| 5.74 ± 2.68 | 8 | Luo Hua An Shen mixture I 1.5% | −3.04 ± 1.75 | 1.78 ± 1.18 |
| 6.06 ± 2.70 | 8 | Luo Hua An Shen mixture I 0.38% | −1.28 ± 0.95 | 3.4 ± 2.59 |
| 6.94 ± 1.96 | 8 | Luo Hua volatile component 10% | 0.125 ± 0.60 | 5.48 ± 1.89 |
| 6.33 ± 3.46 | 8 | Luo Hua volatile component 5% | 0.06 ± 0.3 | 6.44 ± 3.0 |
| 5.91 ± 1.92 | 8 | Danshen injection 6% | −0.63 ± 0.76 | 2.16 ± 0.62 |
| 7.66 ± 2.12 | 8 | Danshen injection 3% | 0.19 ± 1.71 | 2.35 ± 2.36 |
| 6.96 ± 2.78 | 8 | Danshen injection 1.5% | 0.31 ± 1.28 | 4.05 ± 2.36 |

As shown in the Table 7 that the contractile amplitudes induced by 10⁻³M phenylephrine was similar in different groups. Basal arteries were relaxed by Luo Hua An Shen mixture I, in a manner of dose dependant, The contractile activity of 10⁻³M phenylephrine was inhibited by Luo Hua An Shen mixture I significantly. If volatilizable component of Luo Hua An Shen mixture was added, the basal arteries weren't relaxed even in the concentration of 5%, or 10%. Giving phenylephrine again, the basal arteries contracted remarkably as described above. Relaxation of arteries was not observed when Danshen injection was administrated in a concentration 1 time over that of Luo Hua An Shen mixture I. The basal arteries contracted much obviously than that of Luo Hua An Shen mixture I when using phenylephrine again after the treatment of Danshen injection. It could be confirmed that non-volatile component of Luo Hua An Shen mixture I is useful in relaxing the isolated pig basal artery, whereas volatile component and the Danshen injection have no such effects. However, the laxative action of Luo Hua An Shen mixture I on isolated pig basal artery still need to be further testified in vivo.

### Experiment Example 4

Effect of the Present Preparation Containing Leaf Extract of A. hypogaea on Immunological Function in Mice.

As the preparation containing leaf extract of A. hypogaea (Luo Hua An Shen mixture) was effective on the cerebral vessels sclerosis and insomnia, it may be related to enhancing the immunological function. The results of the study are as follows:

1. Influence on Nonspecific Immunological Function in Mice

(1) Immune Organs Weight Assay

Organ weights of Spleen, thymus and lymph node in animals are increased by most of the immunopotentiating agents, but decreased by immunosuppressive agents. So the assay was used to investigate the effect of the preparation containing leaf extract of A. hypogaea on immunological function.

Materials and Methods:

Animals: male and female (1:1) Kunming mice (provided by Shanghai Bioproduct Institute) were used.

Drugs: leaf extract of A. hypogaea, provided by Shanghai Zhoaxiang Chinese Medicinal Herbs Factory. Injection of cortisone acetate was provided by Shanghai Ninth Pharmaceutical Factory.

Methods: The animals were divided into groups at random after weighting. Mice were administered with 84.8 g/1 kg body weight diluted leaf extract of A. hypogaea (in terms of crude drugs) orally once per day for 7 days. 50 mg/1 kg cortisone acetate was injected intramuscularly (totally 3 times), on 2, 4, 6 days after the administration of leaf extract of A. hypogaea. 0.2 ml/10 g body weight physiological saline was administrated orally in control group, once per day for 7 days. Animals were sacrificed on the day 8 by bleeding from orbit vein. Then, spleen, thymus and lymph node (jaw, axillary fossa, groin) were taken out and weighed immediately. Each organic weight was shown as mg/10 g body weight. The results were shown in Table 8, Table 9 and Table 10.

TABLE 8
Spleen weight assay

| | | weight of spleen | |
|---|---|---|---|
| Group | n | (mg/10 g body weight) | P value |
| S.C. | 11 | 4.11 ± 0.63 | 0 |
| cortisone | 10 | 2.58 ± 1.00 | P < 0.01 |
| Leaf of A hypogaea | 10 | 4.70 ± 1.04 | P > 0.05 |

TABLE 9
Thymus weight method

| | | thymus weight | |
|---|---|---|---|
| Group | n | (mg/10 g body weight) | P vaiue |
| S.C. | 11 | 4.40 ± 1.1 | |
| cortisone | 10 | 3.77 ± 0.83 | P < 0.05 |
| Leaf of A hypogaea | 10 | 6.60 ± 1.58 | P < 0.01 |

TABLE 10
Lymph node weight method

| | | lymph node weight | |
|---|---|---|---|
| Group | n | (mg/10 g body weight) | P value |
| S.C. | 11 | 0.14 ± 0.06 | |
| cortisone | 10 | 0.12 ± 0.05 | P > 0.05 |
| Leaf of A hypogaea | 10 | 0.20 ± 0.06 | P < 0.01 |

From the results above, it was shown that leaf extract of A. hypogaea have the effects on increasing the weight of thymus and lymph node.

(2). Carbon Particulate Clearance Test

This method was used to observe the phagocytosis of mononuclear macrophage. Immunopotentiators can activate the macrophage of mice, and enhance the phagocytosis thereof. In contrast, immunosuppressants suppress these effects. Therefore, K values were increased or decreased by these two kinds of drugs, respectively

Animals and drugs were same to those used above.

Methods: After oral administration of drugs or physiological saline, or intramuscular injection of hydrocortisone, for seven days, 0.05 mL/10 g body weight injected through tail vein in mice on day 8. 20 μL blood was acquired from orbit vein, in 1 and 5 minutes after injection, added to 0.1%Na₂CO₃ respectively, and shaken up. Optical density was detected with Model-72 spectrophotometer at λ=680 nm. K values were calculated. The results were statistically analyzed and p values were assessed by t-test. The results were shown in Table 11.

TABLE 11
Carbon participate clearance experiment

| | | weight of spleen | |
|---|---|---|---|
| Group | n | (mg/10 g body weight) | P value |
| S.C. | 11 | 0.00629 ± 0.02319 | |
| cortisone | 10 | 0.00587 ± 0.0033 | P > 0.05 |
| Leaf of A hypogaea | 10 | 0.00739 ± 0.00184 | P > 0.05 |

From the result above, it was obvious that leaf extract of A. hypogaea can not enhance the phagocytosis of mononuclear Macrophage.

2. Mouse Specific Immune Rosette Formation Cell (RFC) Method

Peripheral blood of mouse, spleen cells rosette formation method. This method was used to observe the effect of drugs on antigen combining cell during the earlier stage of immune, which was used to screen immunopotentiator or immunosuppressant. 7.5 mg cyclophosphamide (manufactured by Shanghai Twelve Pharmaceutical Factory) per 1 kg of body weight of male mouse was injected intraperitoneally. According the recommended method for screening immune drugs disclosed by Mr. Lin Zhi-Bing, each group was administrated and immured by injection sheep red blood cell (SRBC). After 4 days, peripheral blood and spleen cell suspension (approximately 9 million spleen cells per milliliter) were obtained from mice, and 1% SRBC and 0.1 mL inactivated calf blood serum were added thereto, intermixed and centrifuged under low-speed for 10 minutes. Then 1% toluidine blue was added in minor amount along the wall of the test tubes. Cell pellet was slightly dispersed by pipette. Well-mixed cell suspension was dropped to blood cell counting chamber and the number of rosette in 1 mm³ was counted under a microscope. Once lymphocyte was surrounded by more than five SRBC, it was regarded as a rosette. Rosette percent was counted in Table 12 and Table 13.

TABLE 12
Peripheral blood rosette test in mice

| Group | n | rosette percent | P value |
|---|---|---|---|
| S.C. | 11 | 20.72 ± 3.98 | |
| cyclophosphamide | 7 | 15.93 ± 3.35 | P < 0.05 |
| Leaf of A hypogaea | 10 | 30.85 ± 7.21 | P < 0.01 |

TABLE 13
Spleen cell rosette test in mice

| Group | n | rosette percent | P value |
|---|---|---|---|
| S.C. | 11 | 20.64 ± 4.8 | |
| cyclophosphamide | 10 | 20.50 ± 4.29 | P > 0.05 |
| Leaf of A hypogaea | 10 | 24.59 ± 3.44 | P < 0.05 |

The results above demonstrated that the leaf extract of A. hypogaea enhanced the rosette formation of peripheral blood and spleen cell of mice. It is considered that leaf extract of A. hypogaea have the action for enhancing specific immunological function compared with saline group.

In conclusion, leaf extract of A. hypogaea was able to increase the weight of spleen, thymus and lymph node, enhance peripheral blood and spleen cell rosette formation in mice. Therefore, it has the effect on enhancing nonspecific immune and cellular immunity.

### Experiment Example 5

Effects of the Present Preparation Containing Leaf Extract of A. hypogaea (Luo Hua An Shen Mixture) on Learning and Memory Ability of Aged Rats

1. Experiment animals: 15 months old male SD rats were random divided into control group and treatment group according to the correct response ratio determined in three times prior to formal training and learning. Except that both groups were fed with same food, water was supplied to control group while 10 mL/rat drugs in water was supplied to treatment group every day. Animals of two groups were trained and learned formally after three weeks.

2. Experimental Method: A labyrinth in the shape of Y with three equal arms (Zhangjiagang Biological and Medical Instrument Factor) was used. Copper sticks with 0.2 cm diameter, 14 cm length were placed with interval of 1 cm in the bottom of labyrinth box with 15 W, signal lights fixed on both ends of the arms which is 45 cm in length.

Learning ability test: The test was performed at the same time every day with 7 days as a cycle. Rat was put into one arm of the labyrinth box acclimatized for 2 minutes. Then electric stimulation was delivered, at the same time the signal lights on another wall clockwise were turned on to indicate where is the safe area (electric stimulation was not provided). If the rat escaped to safe area directly after the stimulation, it was recorded as correct response. Otherwise, it was recorded as wrong response. The process was repeated once again at intervals of 5 seconds. The correct response rate was calculated after 20 times repeat.

Memory ability test: The correct response rate was tested in the same way, on 15, 30, 45 and 60 days after learning test was finished.

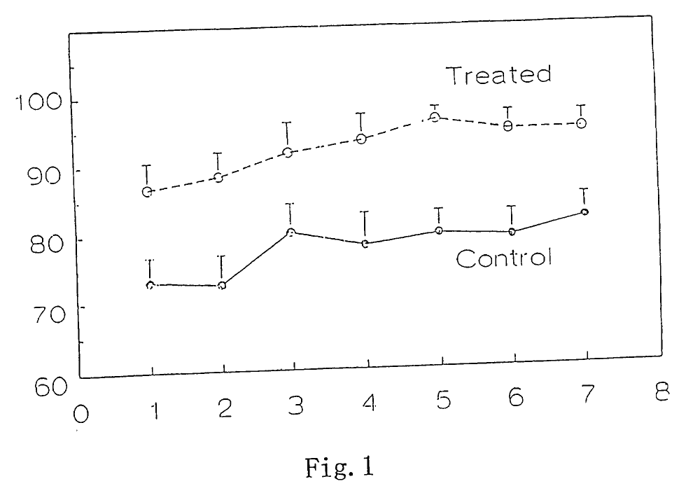

Effect of the present preparation containing leaf extract of A. hypogaea on learning and memory ability of aged rats was shown in FIG. 1.

### Experiment Example 6

The Acute Toxicity Studies on the Present Preparation Containing Leaf Extract of A. hypogaea

Animals: Male and female (1:1) Kunming mice were used, wherein the body weight of the mouse is 18-22 g.

Drugs: 891 An Shen oral liquid (Luo Hua An Shen mixture), choosing the maximum dose and maximum volume which was 5.54 g per 20 g body weight (277 g/kg) in terms of crude drugs, was delivered orally once only. Said dose is 277 times to that used for human (calculated by per kg body weight). No drugs were delivered to the 5 animals in the control group

Observation records: Spontaneous activity of mice in treatment group was reduced than that of pre-administration of drugs. But they were still able to move and had the exploratory and adjunct behavior. Lethargy was not observed. Activity was recovered to the pre-administration level after one hour.

The mice were observed for 7 days, with normal eating, free activity, glossy fur, and increased body weight. Further, none was dead. A pathological anatomy was conduced after 7 days. Activity of mice in control group was normal.

Results: 5 male mice in control group (no treatment) were observed

Heart: Serous membrane was pink, left-right auricle and ventricle were dilated obviously. Cardiac valves were completeness.

Lung: Left and right lungs were pink without visible edema or hyperemia.

Kidney: The surface of left and right kidneys was smooth and glossy, pink, without obvious tumefaction or hyperemia.

Liver: the surface was smooth and glossy, pink, without tumefaction or hyperemia.

Spleen: the surface was smooth and glossy, pink, without tumefaction or hyperemia. 10 male and 10 female mice in treatment group were observed compared with those in control group

Heart: The size of heart was similar to that in control group. Serous membrane was pink, left-right auricle and ventricle were not dilated obviously. Cardiac valves were completeness.

Lung: Left and right lung were pink, without visible edema or hyperemia.

Kidney: The size of left and right kidneys was similar to that in control group. The surface of left and right kidneys was smooth and glossy, without obvious tumefaction or hyperemia.

Liver: The size of liver was similar to that in control group. The surface was smooth and glossy, pink, without tumefaction or hyperemia.

Spleen: The surface of it was smooth and glossy, pink, without tumefaction or hyperemia.

### Experiment Sample 7

The Long-term (Chronic) Toxicity Studies on the Present Preperation Containing Leaf Extract of A. hypogaea (Luo Hua An Shen Mixture) by Oral Administration in Rats

Drug: Luo Hua An Shen mixture.

Animals: 6 weeks old SD rats with body weights of 80-120 g were provided by B-K Company of Shanghai Birth Control Technique Institute. After fed for 1 week, the body weights increased to 116-155 g.

Method: Animals were fed for 1 week in the room with constant temperature (20±1° C.). Physiological and biochemical parameters were determined. 60 animals with normal parameters were chosen by weight and random divided into three groups, that is, each group included 20 mice with equal numbers of male and female animals. The two test groups were orally delivered with 120 and 30 g/kg Luo Hua An Shen mixture (in terms of the crude drugs thereof), which is 100 and 25 times to those used in clinic respectively (planning clinical dose was 60 g/50 kg body weight, viz. 1.2 g/kg) Control group animals were administrated orally with 10 mL/kg distilled water with the equal volume to that used in treatment groups. Drugs and water were delivered once per day (except Sunday) for three months. Half of the animals were killed for pathological examination after drugs withdraw. The rests were killed for pathological examination after continued observation for another month. The observations of general condition in rats included behavior, food and water intake, body weight and stool etc. Body weight was determined once a week. Food intake was recorded every day. The hematological parameters included RBC, HB, WBC, and DC. The blood biochemical parameters included AST, ALT, BUN and Tch The parameters above were determined at the time before administration (d₀), during administration (d₄₅, d₉₀) and in a month after withdraw (d₁₂₀), respectively. The organs for pathological examination included heart, lung, stomach, intestine (colon, ileum, duodenum), liver, pancreas, spleen, kidney, brain, bladder, testis, epididymides (or uterus, ovaries), prostate, thymus, adrenal gland, mesenteric lymph nodes, spinal cord etc.

The organs were examined macroscopically. Viscera coefficients were calculated and pathological tissue sections were made. If the toxicity related to drug was not been detected with microscope examination in high dosage group and control group, low dosage group would not been examined anymore.

Statistical analyses: Data were illustrated as X±SD. Statistical analyses were performed with F test between the groups. Remarkable difference was judged with P<0.05.

Chronic toxicity study indicated that Luo Hua An Shen mixture given by 25 times and 100 times of planning clinical dose orally for three month in rats, did not show any abnormal changes in physiology, biochemistry and pathology. All of the parameters were in a normal range. Therefore, Luo Hua An Shen mixture by oral administration was substantially non-toxic to rats.

1. A process for preparing the preparation containing leaf extract of Arachis hypogaea, comprising:
drying and pulverizing the stem and leaves of A. hypogaea, passing through sieve of 10-40 meshes;
charging into a multifunctional reaction vessel equipped with heating jacket and reflux decice, in which
144 parts leaves are heated under reflux with 200-1000 parts non-polar solvent for 1˜3 times, 2˜4 hours for each time; or steam distillated to extract the liposoluble component A,
the residual leaves are extracted again with 200-1200 parts polar solvent for 1˜3 times, 1˜4 hours for each time, after removing the solvents,
combine the two kinds of extracted solutions, thus obtain the leaf extract, and
formulating the preparation by mixing 1˜85% said leaf extract with 99˜15% pharmaceutical carrier or excipient based on 100% preparation.

2. A process according to claim 1, wherein extracting and separating of the leaves comprise the steps:
extract the leaves of A. hypogaea twice with adequate amount of water under boiling after pulverizing, and then filter; combine the extracted solutions, and concentrate said combined solution to form a extractum which contains approximately 50% water; mix the extractum with inert material uniformly, triturate after dried under 70° C., then charge the resultant into a Soxhlet's extractor, extract with petroleum ether, ethyl acetate, acetone and ethanol successively, or extract with specifically solvent according to the structure of different fractions of compounds

3. A process according to claim 2, wherein said different fractions of compounds are: A) total extract; B) protein; C) polysaccharide; D) flavone, tannin; and E) liposoluble substance, volatile oil.

4. A process according to claim 1, wherein the non-polar solvent is selected from ether and petroleum ether.

5. A process according to claim 1, wherein the polar solvents is selected from ethanol and water.

6. A preparation containing leaf extract of A. hypogaea prepared by the process according claim 1.

7. A preparation according to claim 6, which is oral liquid.

8. A preparation according to claim 6, which is capsule.

9. A preparation according to claim 6, which is tablet

10. A preparation according to claim 6, which is granule.

11. A preparation according to claim 6, which is diluent.

12. A preparation according to claim 6, which is powder.
